(19) United States
(12) Patent Application Publication
(10) Pub. No.: US 2009/0169635 A1
(43) Pub. Date: Jul. 2, 2009

(54) PHARMACEUTICAL COMPOSITIONS AND USE THEREOF

(75) Inventors: Joseph Schwarz, Markham (CA); Michael Weisspapir, Markham (CA); Hai Yan Gao, Markham (CA)

Correspondence Address: ALPHARX INC., 168 KONRAD CRESCENT, SUITE 200, MARKHAM, L3R 9T9, CA

(73) Assignee: AlphaRx Inc., Markham (CA)

(21) Appl. No.: 11/968,084

(22) Filed: Dec. 31, 2007

(51) Int. Cl. A61K 9/51; A61P 31/00

(52) U.S. Cl. 424/497; 977/773

(57) ABSTRACT

Colloidal compositions, loaded with non-covalently bonded antibiotics, can be efficiently used for the treatment of severe bacterial pneumonia and other serious lung infections such as tuberculosis. Such formulations, comprised of biodegradable nanoparticles or nanocapsules with incorporated antibiotics, show a significant increase in antibacterial activity, extended and sustained drug release and a decrease in frequency of the drug administration. Antibiotics of various types, such as aminoglycosides, glycopeptides and others can be successfully incorporated into a nanoparticulate colloidal delivery system.

### BACKGROUND OF INVENTION

With a growing number of bacterial strains which are resistant to traditional antibiotics and the associated development of nosocomial pneumonia, there is an increased need for the development of treatment methods to address these issues. Vancomycin, introduced in mid-1950's, remains a clinically important and effective antibiotic. However, it has several limitations, including a relatively slow bactericidal activity evolving, fluctuated minimum inhibitory concentrations (MICs), the development of resistance and other associated therapeutic failures, poor pharmacokinetic properties and the potential for serious toxicity, as described by D. P. Levine [1]. Vancomycin failure rates among patients with endocarditis, bacteremia, or bacteremic pneumonia due to methicillin-susceptible Staphylococcus aureus (MSSA) or methicillin-resistant Staphylococcus aureus (MRSA) are steadily growing, with reported failure rates from 37% to as high as 50%, as reported in articles by D. L. Stevens [2] and A. M. Ferrara [3].

Nosocomial pneumonia and ventilator-assisted pneumonia may be polymicrobial in nature and can be caused by a wide spectrum of pathogens. Potentially multi-drug resistant microorganisms often represent the ‘core’ pathogens of the most severe infections. Among Gram-positive pathogens, methicillin-resistant Staphylococcus aureus (MRSA) plays a key role, mainly in mechanically ventilated patients or in patients with specific risk factors. The mainstay of treatment for MRSA pneumonia has been glycopeptide antibiotics, i.e. vancomycin and to a lesser extent, teicoplanin. However, owing to poor penetration into lung compartments, vancomycin may result in therapeutic failure or slow clinical responses. Moreover, vancomycin serum levels must be monitored in order to minimize nephrotoxicity and to maximize drug concentration in the lung. Finally, with the emergence of staphylococci isolates with reduced susceptibility to vancomycin, glycopeptides may no longer be the appropriate first-line antibacterial agents for Gram-positive lung infections.

MRSA pneumonia is difficult to treat because of the limited number of effective drugs available. All beta-lactam antibiotics are excluded and many MRSA isolates are also resistant to several other currently used antimicrobials (e.g. macrolides, lincosamides, aminoglycosides, tetracyclines, chloramphenicol and quinolones). Vancomycin, which to date has been the drug of choice for MRSA infections, has only moderate efficacy in pulmonary infections because of its unsatisfactory pharmacokinetic profile in lung tissue, according to M. Cruciani et al. [4]. Penetration of vancomycin into different lung compartments is extremely poor. C. Lamer et al. [5] found that its' concentration in epithelial lining fluid does not exceed 20% of plasma levels, with high inter-individual and intra-individual variations resulting in inadequate concentrations in approximately 40% of patients treated with a standard dosage (1 g twice daily). The degree of serum protein binding of vancomycin appears to hamper adequate penetration of the drug into lung tissue of patients with MRSA pneumonia. This limited tissue penetration, along with an upward drift in the MIC of vancomycin in treating MRSA, offers a convincing rationale for the observed clinical failure of vancomycin in treating patients with serious pulmonary MRSA infections, according to M. H. Kollef [6]. M. D. Kitzis and F. W. Goldstein [7] suggest a further negative characteristic of this drug—the need for constant monitoring of serum vancomycin levels to maintain effective therapeutic serum levels and to reduce the risk of nephrotoxicity.

Among new therapeutic options, linezolid may be an appropriate choice for MRSA pulmonary infections due to its efficacious pharmacokinetic profile in the lung and its acceptable tolerability in patients with renal insufficiency or receiving other nephrotoxic agents. However, to contain the increasing emergence of drug resistance among hospitalized patients, these novel antimicrobial agents must be used judiciously, restricting their use to patients who are not responsive to, or intolerant of, glycopeptides, as described by M. H. Scheetz et al. [8].

Vancomycin's status as the drug of choice in the treatment of methiciliin-resistant Staphylococcus aureus MRSA pneumonia has been called into question on the basis of increasing numbers of therapeutic failures. In patients with MRSA pneumonia, treatment failures are probably due to the complex interplay of variables affecting the host, antimicrobial and pathogen interrelationship. It has been suggested that the decreased penetration of vancomycin into the lung may be a contributing factor as well. This review explores the physiochemical and physiologic variables that affect pulmonary penetration and describes methods used in quantifying pulmonary vancomycin concentrations. Most importantly, findings are evaluated in the clinical context of chemotherapeutic options available for treatment of MRSA pneumonia. The possibility of increasing serum vancomycin concentrations as a method of optimizing current treatment outcomes is also explored.

The intracellular activity of antibiotics is dependent on their pharmacokinetic and pharmacodynamic parameters. Low transmembrane penetration and decreased intracellular activity are the major reasons for the limited activity of most antibiotics (penicillins, cephalosporins, aminoglycosides) in intracellular infections. An additional difficulty, particularly with conventional antibiotic therapy, is that many intracellular bacteria are quiescent or dormant. These bacteria are present in a reversible state and can persist for extended periods of time without division in a viable but non-culturable state. Microorganisms in infected tissues are protected by various biological structures around infection foci. Indeed, the adhesion properties of bacteria are expressed by secreting a glycocalyx in pathological conditions, providing increased protection and hence, increased resistance to antibacterial agents (see R. Eng et al., [9]). Despite the discovery of new antibiotics, the treatment of intracellular infections often fails to eradicate the pathogens. H. Pinto-Alphandary et al. in a review article [10] suggest that by loading antibiotics into colloidal carriers, liposomes and nanoparticles, one can expect improved delivery to infected cells. U.S. Pat. No. 4,897,384 by A. Janof and U. S. Pat. No. 5,759,571 by E. M. Hersch and an article by R. Schiffelers et al. suggest that the use of liposomes loaded with antibiotics, results in higher antibacterial action than antibiotics administered alone, especially in the treatment of intracellular infections [11-13].

In article [14] of P. R. J. Gangadharam et al., Streptomycin 100 mg/kg, given intramuscularly (IM) five days a week for four weeks, results in a significant reduction in bacterial count of Mycobacter avium complex (MAC) from spleen, lungs and liver. Streptomycin given encapsulated in an, multi-lamellar liposomal form at dose 15 mg/kg, in two intravenous (IV) injections causes an even greater reduction in bacterial counts in the three tissues. Comparing free Streptomycin given at 150 mg/kg given IM five days a week for eight weeks with Streptomycin encapsulated in unilamellar liposomes given IV at 15 mg/kg in four injections (day one and at three weekly intervals with no further treatment within the eight weeks), shows a several-fold increase in the chemotherapeutic efficacy of the drug administered in the latter form. Similar results were obtained in publication of Oh Yu-Kyoung, et al. [15] where Mycobacter avium complex infection is treated with liposome encapsulated antibiotics.

Nevertheless, leakage of drug from liposomes during storage limits the development of stable liposomal formulations for the delivery of hydrophilic antibiotics [13-15]. Owing to their polymeric nature, nanoparticles may be more stable than liposomes in biological fluids and during storage. The nanoparticles in the body must be degraded in vivo to avoid side effects due to intracellular polymeric overloading. Polyalkylcyanoacrylate nanoparticles satisfy such requirements, they are extensively studied because of ease of manufacture and appropriate physicochemical properties (see U.S. Pat. No. 4,329,332 to P. Couvreur et al., [16]). They may be freeze-dried and rehydraied without modifying the size and drug content. Their structure allows better retention of the drug inside the polymeric network and then nanoparticles can slowly degrade by esterase action. Alkylcyanoacrylates with long side chains are preferred (see article of K. S. Soppimath et al. [17]) since the acute toxicity of these polymers is greatly reduced.

Biodegradable polymeric NP's have recently attracted considerable attention as potential drug delivery devices in view of their applications in the controlled release (CR) of drugs, their organ/tissue specific targeting, as carriers of DNA in gene therapy and in their ability to deliver proteins and peptides.

The majority of these NPs are preparations made of polyesters of hydroxyacids: poly(D,L-lactide)-poly(lactic acid) PLA, poly(D,L-glycolide), PLG, poly(lactide-co glycolide), PLGA, poly(e-caprolactone), PCL and poly(cyanoacrylate) PCA, as well as NPs based on hydrophilic polymers—chitosan, gelatin, sodium alginate and other.

The PLA, PLG and PLGA polymers are tissue-compatible and have been extensively used as microparticulate sustained-release formulations in parenteral and implantation drug delivery applications. Poly(e-caprolactone), PCL, and poly(alkylcyanoacrylates), PACA, are also used in NP preparations.

The research group of P. Couvreur et al. [16, 18] described nanoparticles of polyalkylcyanoacrylates loaded with Ampicillin and other antibiotics. These polymers are bioresorbable and have been in use for several years as surgical glues. Ampicillin, incorporated into PIHCA NP, is more than 100 times as effective as free drug in salmonellosis treatment, according to E. Fattal et al. [18]. This higher efficacy of nanoparticle-bound Ampicillin is observed in the treatment of experimental acute murine salmonellosis and chronic Listeria monocytogenes infections. This is attributable to the combined effect of two types of drug targeting. Firstly, as demonstrated by tissue distribution studies, the binding of Ampicillin to nanoparticles leads to a high concentration of drug in the liver and spleen, major foci of infection. Secondly, there is a much higher cellular uptake by macrophages of Ampicillin bound to nanoparticles than in the free form. The uptake of nanoparticles by an endocytotic mechanism allows intra-lysosomal localization of carrier and a subsequent increase in the intracellular concentration of the targeted drug. This suggests that ampiciliin-bound nanoparticles may be very effective in the treatment of intracellular bacterial infections in animals and humans.

Colloidal delivery systems such as nanoparticles are extensively absorbed within the reticulo-endothelial (REM) system of the body, mainly by mononuclear phagocytes, and are thus quickly eliminated from blood circulation. Cellular absorption can be inhibited by coating nanoparticles with hydropbiiic polymers such as PEG derivatives, masking particles from cellular internalization and increasing their circulation time [13, 16]. Some polymers can modify the opsonization process, altering the targeting of particles, as described in paper of D. E. Owens et al. [19] and in U.S. Pat. No. 7,025,991 to B. A. Sabel et al. [20]

### SUMMARY OF THE INVENTION

It is well known that parenterally administrated colloidal preparations are actively absorbed by the reticulo-endothelial system (RES) organs (macrophages, lymphocytes, Payer patches, liver, spleen) and thus eliminated from the blood circulation. Such behavior makes nanoparticulate formulations, liposomes or submicron emulsions, less effective in the treatment of infectious diseases with foci outside of the RES, e.g., pneumonia, cystic fibrosis or meningitis.

Nevertheless it was surprisingly found that biodegradable polymeric nanoparticles, loaded with water soluble antibiotics, can efficiently cure infections of lungs even without use of “stealth” technology and providing extended circulation of the colloidal particles in the blood.

This invention is intended to treat bacterial lung infections such as pneumonia, tuberculosis and other, using a systemically administered drug delivery system which is composed of biodegradable polymer nanoparticles with an incorporated antibiotic.

More particularly, the present invention is directed to a treatment of infections, caused by Staphylococcus aureus, Enterobacter faecis, Mycobacter tuberculosis, Klebsiella, Streptococci, Clostridia, Brucella, Acinetobacter and others by systemic administration of a nanoparticulate drug-delivery system, comprising antibacterial drug. In accordance with an important aspect of the present invention, the drug is water soluble and association of the drug with polymeric nanoparticles is between 5 to 100%, preferably 10 to 80% of total amount of the antibiotic. Preferred drugs are antibiotics, selected from classes of aminoglycosides, peptides and glycopeptides, e.g., Streptomycin, Gentamicin, Kanamycin, Vancomycin, Polymixin, Colistin, etc.

Another aspect of the present invention is to provide a nanoparticulate drug composition wherein the biodegradable polymer is a polyester-type polymer, such as polylactide (PLA), polyglycolide (PGA), lactide-glycolide copolymer (PLGA), polycaprolactone (PCL), poly(hydroxy)butyrate (PHB) or a combination of such polymers with biocompatible lipids and hydrophobic compounds, such as phosphatidylcholines, mono-, di- and triglycerides, waxes, aliphatic or aromatic esters.

Yet another aspect of the present invention is to provide a pharmaceutical composition with enhanced antibacterial action, comprised of biodegradable nanoparticles loaded with an antibacterial drug which is administered to an individual in a quantity that is therapeutically effective in treating an acute or chronic disease or condition and wherein the cumulative amount of drug in nanoparticulate composition is several times lower than the dosage of a conventional drug formulation.

Another aspect of the present invention is to provide a pharmaceutical preparation comprising biodegradable nanoparticles containing a water soluble drug that remain associated with nanoparticles immediately after administration, and that are capable gradually release the incorporated drug in vivo for extended period of time to heat infection disease or conditions, associated with bacterial infections.

One other aspect of the present invention is to increase the binding of the water soluble antibiotic to hydrophobic nanoparticles, thus providing improved safety, diminished side-effects and prolonged sustained release of the composition.

Controlled delivery of an antibacterial drug by means of a biodegradable and biocompatible nanoparticulate delivery system offers profound advantages over conventional antibiotic delivery. Drugs can be used more effectively and efficiently, less drug is required for optimal therapeutic effect and toxic side effects can be significantly reduced or eliminated through drug targeting. The stability of some drags can be improved, allowing for a longer shelf-life and drugs with a short half-life can be protected within the nanoparticulate matrix from destruction, thereby ensuring sustained release of the active agent over time. The benefit of a continuous, targeted release of drug includes sustained drug levels within a constant therapeutic range and drug presentation either continuously or in a pulsatile mode, as required, to obtain an optimal therapeutic outcome. All of these effects can be accomplished with significantly reduced number of, or even single dose administrations of encapsulated drug.

Due to low toxicity and high biocompatibility of PLA, PLG, PCL, and PLGA polymers, these materials are used for preparation of colloidal delivery system for targeting of antibiotics after parenteral administration.

Unexpectedly, incorporation of antibiotics into nanoparticles having much slower degradation rate, compared with liposomes and polyalkylcyanoacrylates, significantly increasing their relative antibacterial activity and providing a substantial decrease in the cumulative dose of administered drug.

NP formulations, prepared according to the invention, were tested in animals infected with virulent strains of bacteria causing severe illness. As anticipated, the mortality rate, cumulative antibiotic dose and frequency of drug administration for NP formulations are significantly lower than for standard treatment protocols for various antibiotics used in a variety of animal disease models.

### DETAILED DESCRIPTION OF INVENTION

### Nanoparticles Preparation

Nanoparticles with incorporated antibiotics are prepared by double emulsion technique or by nanoprecipitation at different drug-to-polymer ratios and water soluble coadjuvants are added to the water phase in various concentrations. After the elimination of organic solvents, a suspension of the formed nanoparticles is concentrated and filtered through a microporous filter membrane. The particle size is measured by photon correlation spectroscopy (Malvern Zetasizer “Nano-S”). For evaluation of drug loading in NP, free drug is separated by ultrafiltration (separation membrane with molecular cutoff 30,000 or 300,000 NMWL) and its concentration is measured by HPLC.

### Streptomycin in Biodegradable Polymeric Nanoparticles

### EXAMPLES 1-9

50-500 mg of antibiotic (Streptomycin sulfate USP) was dissolved in 0.5-1.0 ml of purified water and emulsified in 5-10 ml of organic solvent (Ethyl Acetate, saturated with water, dry chlorophorm or pure methylene chloride), containing dissolved D,L-lactide-glycolide copolymer (e.g., Resomer® 502, 503 or 503H, Boehringer Ingelheim, Germany), with the help of short sonication (30 sec) at 20 kHz using titanium indenter or a high shear rotor-stator mixer (Ultra-Turrax® T10, IKA, Germany). The formed emulsion was added to a continuous water phase, containing surfactants and may contain other water soluble adjutants, and further homogenized (30 sec sonication, 3-5 cycles of high pressure homogenization (Avestin Emulsiflex® C5 or similar machine). The fine emulsion thus obtained was evaporated under decreased pressure (2-100 mm Hg) to eliminate organic solvents and concentrate the product to a final volume of 10 mL. The final suspension of nanoparticles was centrifugated (10 minutes, 1000 g) to remove aggregates, and filtrated through 0.45 mcm microporous membrane. The particle size and size distribution was measured by photon correlation spectroscopy (Malvern Zetasizer® Nano-S) in water. For evaluation of the drug associating with NP, a clear drug solution

was separated by transmembrane uitracentrifugation (separation membrane with

molecular cutoff 30,000 or 300,000 NMWL) and antibiotic concentration was determined by HPLC.

TABLE 1
Streptomycin loaded polymeric nanoparticles

| Example #: | 1 | 2 | 3 | 4 | 5 | 6 | 7 | 8 | 9 |
|---|---|---|---|---|---|---|---|---|---|
| Streptomycin | 150 | 150 | 150 | 50 | 50 | 150 | 50 | 50 | 50 |
| sulfate, mg | | | | | | | | | |
| Polymer | 503H | 503H | 503H | 502S | PCL 10K | 503H | 503H | 503H | 504H |
| Polymer, mg | 500 | 500 | 500 | 400 | 400 | 400 | 400 | 400 | 400 |
| Surfactant(s) | TPGS 0.5% | Cremophor | Cremophor | BSA 3% | TPGS 3% | TPGS 2% | TPGS 2% | TPGS 2% | TPGS 1% |
| | Solutol | EL 2% | EL 2% | | | | Lipoid | Lipoid | BSA 2% |
| | HS15 1% | | Lipoid | | | | 75SA 0.5% | S80H 0.4% | |
| | | | S80H 0.5% | | | | | | |
| Counter-ion | | | | | Sodium | | | | |
| | | | | | Deoxycholate | | | | |
| | | | | | 0.5% | | | | |
| Particle size, nm | 241 | 217 | 151 | 140 | 72 | 187 | 103 | 91 | 163 |
| Binding | 29% | 31% | 48.5% | 9.8% | 88% | 33.9% | 20.0% | 20.9% | 42.2% |

### Vancomycin in Biodegradable Polymeric Nanoparticles

### EXAMPLES 10-15

Vancomycin loaded polymeric nanoparticles were obtained by a method similar to that described earlier in ex. 1-10. Ethyl acetate was used as an organic solvent, D,L-(poly(lactic)-poly(glycolic) block copolymer Resomer® RG from Boehringer Ingelheim was used as a matrix material of nanoparticles. Drug binding estimation was carried out by transmembrane uitracentrifugation or by sedimentation of the nanoparticles

by high speed centrifugation. The final volume of the product—10 ml, results are shown

in Table 2.

TABLE 2
Vancomycin loaded polymeric nanoparticles

| Example #: | 10 | 11 | 12 | 13 | 14 | 15 |
|---|---|---|---|---|---|---|
| Vancomycin | 100 | 100 | 100 | 100 | 100 | 100 |
| sulfate, mg | | | | | | |
| Polymer | 502H | 502H | 502H | 502H | 502S | 503H |
| Polymer, mg | 400 | 400 | 400 | 400 | 400 | 400 |
| Surfactant(s) | Tween 80 2% | Tween | Tween | Cremophor | Tocophersolan 1% | Tween 80 2% |
| | Lipoid | 80 2% | 80 2% | EL 2% | Lipoid S80 0.5% | Lipoid |
| | S80H 0.5% | | | | | S80 0.5% |
| Counter-ion | | Tocopherol | Cholesterol | | | |
| | | succinate | sulfate | | | |
| Particle size, nm | 121 | 182 | 71 | 131 | 130 | 117 |
| Binding | 19% | 12% | 73.3% | 8.5% | 15.8% | 5% |

### Polymixin B in Biodegradable Polymeric Nanoparticles

### EXAMPLES 16-23

Polymixin B loaded polymeric nanoparticles were obtained by a method similar to that described earlier in ex. 1-10. Ethyl acetate was used as organic solvent, D,L-(poly(lactic)-poly(glycolic) block copolymer Resomer® RG from Boehringer Ingelheim was used as a matrix material of nanoparticles. Various counter-ions were used to improve drug incorporation into nanoparticles. Drug binding estimation was carried on by transmembrane uitracentrifugation or by sedimentation of the nanoparticles by high speed centrifugation. The final volume of the product—10 ml, results are shown in Table 3.

TABLE 3
Polymixin B loaded polymeric nanoparticles

| Example #: | 16 | 17 | 18 | 19 | 20 | 21 | 22 | 23 |
|---|---|---|---|---|---|---|---|---|
| Polymixin | 10 | 10 | 10 | 10 | 10 | 25 | 25 | 25 |
| sulfate, mg | | | | | | | | |
| Polymer | 502 | 502 | 502 | 502 | 502S | 502 | 502 | 503H |
| Polymer, mg | 200 | 200 | 200 | 200 | 200 | 200 | 200 | 200 |
| Surfactant(s) | Tween | Tween | Tween | Tween | Tween | Tween | Tween | Pluronic |
| | 80 2% | 80 2% | 80 2% | 80 2% | 80 2% | 80 2% | 80 2% | F-68 |
| | Lipoid | | | | Lipoid | | | |
| | S80 0.5% | | | | S80 0.5% | | | |
| Counter-ion | | Naphtyl- | Vit. E | Stearic | Sodium | Vit. E | Vit. E | Vit. E |
| | | sulfonic | succinate | acid | caprylate 0.1% | succinate | succinate | succinate |
| | | acid | | | | 0.25% | 0.5% | 0.5% |
| Particle size, nm | 92 | 90 | 213 | 86 | 130 | 102 | 129 | 87 |
| Binding | 17.6% | 12% | 15% | 11% | 15.8% | 13.6% | 26.8% | 27% |

### Colistin (Polymixin E) in Biodegradable Polymeric Nanoparticles

### EXAMPLES 24-31

Colistin loaded polymeric nanoparticles were obtained by a method similar to that described earlier in ex. 1-10. Ethylacetate was used as organic solvent, D,L-(poly(lactic)-poly(glycolic) block copolymer Resomer® RG from Boehringer Ingelheim was used as a matrix material of nanoparticles. Various counter-ions were used for improvement of drug incorporation into nanoparticles. Drug binding estimation was carried out by transmembrane uitracentrifugation. The final volume of the product—10 ml, results are shown in Table 4.

TABLE 4
Colistin loaded polymeric nanoparticles

| Example #: | 24 | 25 | 26 | 27 |
|---|---|---|---|---|
| Colistin | 20 | 20 | 20 | 20 |
| sulfate, mg | | | | |
| Polymer | 503H | 503H | 503H | 503H |
| Polymer, mg | 200 | 200 | 200 | 200 |
| Surfactant(s) | Tween | Tween 80 1% | Tween 80 1% | Tween 80 1% |
| | 80 1% | Lipoid S80H 0.5% | | |
| Counter-ion | | Tocopherol acid | Cetyl- | Oleic acid |
| | | succinate 0.5% | phosphate | |
| Particle | 263 | 157 | 164 | 73 |
| size, nm | | | | |
| Binding | 12.8% | 18.6% | 23.2% | 6.2% |

### Amicacin in Biodegradable Polymeric Nanoparticles

### EXAMPLES 28-33

154-167 mg of antibiotic (Amikacin sulfate USP, equal to 100 mg Amikacin base) was dissolved in 0.3-0.5 ml of purified water and emulsified in 5-10 ml of organic solvent (water saturated Ethyl Acetate), containing dissolved D,L-lactide-glycolide copolymer (Resomer®, Boehringer Ingelheim, Germany) with help of short sonication. The formed emulsion was added to a continuous water phase, containing surfactants and may contain other water soluble adjutants and further homogenized (30 sec. sonication, 5 cycles of high pressure homogenization (Avestin Emulsiflex® C5 at 12,000-18,000 psi). The obtained fine emulsion was evaporated under decreased pressure (2-100 mm Hg) to eliminate organic solvents and concentrate the product to a final volume of 10 mL. The final suspension of nanoparticles was centrifuged (10 minutes, 1000 g) to remove aggregates, and filtered through a 0.45 mcm microporous membrane. The particle size and size distribution was measured by photon correlation spectroscopy (Malvern Zetasizer® Nano-S) in water. For evaluation of the drug associating with NP, a clear drug solution was separated by transmembrane ultracentrifugation (separation membrane with molecular cutoff 300,000 NMWL) and antibiotic concentration was determined by HPLC. The final volume of the product—10 ml, results are shown in Table 5.

TABLE 5
Amikacin loaded polymeric nanoparticles

| Example #: | 28 | 29 | 30 | 31 | 32 | 33 |
|---|---|---|---|---|---|---|
| Amikacin | 167 | 154 | 154 | 154 | 154 | 154 |
| sulfate, mg | | | | | | |
| Polymer | 503H | 503H | 503H | 503H | 503H | 503H |
| Polymer, mg | 600 | 400 | 400 | 400 | 400 | 400 |
| Surfactant(s) | Tween | Tween | Cremophor | Tween | Tween | Tween |
| | 80 2% | 80 2% | EL 2% | 80 2% | 80 5% | 80 2% |
| | Lipoid | Lipoid | Lipoid S80 | | | |
| | S80H 0.5% | S80H 0.5% | 0.5% | | | |
| Counter-ion/lipid | | | | Vit. E | Oleic | Cholesterol |
| | | | | succinate | acid | sulfate |
| | | | | 0.5% | | |
| Particle size, nm | 286 | 157 | 201 | 202 | 116 | 207 |
| Binding | 14.2% | 18.6% | 35.6% | 35.1% | 30% | 32.8% |

### Improved Treatment of Lung Infectious Disease With Antibiotic Loaded Nanoparticles

### Tuberculosis

An investigation of the antituberculosis efficacy of nanoparticulate forms of Streptomycin for parenteral administration was carried out in comparison with conventional Streptomycin sulfate for injections.

### Bacteria

Mycobacterium tuberculosis strain H₃₇RV (ATCC 27294) bacteria were grown to mid log phase in Difco™ broth, supplemented with albumin-dextrose-catalase and 0.05% Polysorbate 80. Cultures are incubated at 37° C. with 5% CO2 and continuous shaking. Bacteria are harvested by centrifugation at 5,000 g, washed with sterile pyrogen-free saline, concentrated to 3×10⁸ CFU/ml, aliquoted and stored at −70° C. until use. Aliquots are thawed before inoculation.

### Animals

Specific-pathogen-free (SPF) BALB/C female mice, weighing 18-22 g., not more then six per cage, were housed in an air-conditioned biohazard room designed for infectious animals model, with temperature 21±2° C., humidity 55±15% and a 12-hour light/12-hour dark cycle. They have access to food and filtered tap water ad libitum. Mice were acclimatized in the animal facilities for 1 week prior to the commencement of the experiment.

### Bacterial Inocula

For inoculation, the frozen stock is thawed, diluted to concentration of 10⁸ CFU/mL with sterile pyrogen free 0.9% solution of Sodium chloride for injection containing 0.01% Polysorbate 80 (sterile filtered in aseptic conditions) and sonicated for 2 minutes to disperse clumps.

Each mouse is inoculated intravenously (lateral tail vein) with 10⁷ mycobacteria in a volume of 0.1 mL. The amount of bacteria used as the inoculation dose was verified retrospectively by serial dilution on Middlebrook 7H10 agar, supplemented with oleic acid-albumin-dextrose-catalase.

At Day 0 (D0) all mice are inoculated intravenously with 10⁷ M tuberculosis per mouse. The next day (D1) after bacterial inoculation, 6 infected animals (Baseline Control 1) were sacrificed to provide baseline values of body, spleen and lung weight, lung lesions, and the number of CFU in the spleen and lungs (bacterial count).

The remaining mice were divided randomly into experimental groups (18 animals in each group). Each experimental group was subdivided into two subgroups of 6 and 12 animals. The first animal subgroup was sacrificed at D28 and the second subgroup was monitored for mortality daily as to lethality for a period of 8 weeks. All surviving animals were euthanized at D56 to perform terminal procedures.

### Experimental Treatment (Dosing)

Treatment with antibiotic is started the day after bacterial inoculation at Day 1 (D1) and carried out for 4 weeks (28 days), followed by an additional 28 days of observation. Animals from Positive Control and Comparison Control groups were treated with Streptomycin sulfate for injection (conventional form). The route of administration for all experimental groups is intraperitoneal injection, in a dose corresponding to 200 mg/kg of Streptomycin base.

The baseline control group receives no treatment.

The positive Control group received intraperitoneal injections of Streptomycin sulfate in a dosage equal to 200 mg/kg of Streptomycin base, 5 times a week for 4 weeks, to a total cumulative dose of 4000 mg/kg.

The comparative control group received intraperitoneal injections of Streptomycin sulfate in a dosage equal to 100 mg/kg of Streptomycin base twice a week for 4 weeks, to a total cumulative dose of 800 mg/kg.

Nanoparticulate formulations were administrated in a dosage equal to 100 mg/kg of Streptomycin base, twice weekly for 4 weeks.

Experimental results are presented in tables 6 and 7 and graph 1.

TABLE 6
Survival rate after treatment of the mice, infected with Mycobacterium tuberculosis,
with different Streptomycin preparations

| | | | Cumulative dose | | | | |
|---|---|---|---|---|---|---|---|
| | Dose | Frequency of | of SM base, | Numbers | Survival rate (%) | | |
| Groups | (mg/kg) | administer/week | mg/kg | of deaths | 14 d | 28 d | 56 d |
| Untreated (Control group) | 0 | 5 | 0 | 12/12 | 0 | 0 | 0 |
| Positive control (Streptomycin | 200 | 5 | 4000 | 1/12 | 92 | 92 | 92 |
| USP, 200 mg/kg, 5 × week, 4 | | | | | | | |
| weeks, totally 4000 mg/kg) | | | | | | | |
| Comparative control | 100 | 2 | 800 | 5/12 | 83 | 58 | 58 |
| (Streptomycin USP, 100 mg/kg, | | | | | | | |
| 2 × week, 4 weeks, totally 800 | | | | | | | |
| mg/kg) | | | | | | | |
| NP formulation (Example # 4) | 100 | 2 | 800 | 2/12 | 83 | 75 | 75 |
| NP formulation (Example # 3) | 100 | 2 | 800 | 0/12 | 100 | 100 | 100 |

Mycobacterium tuberculosis and Treated With Different Streptomycin Preparations

TABLE 7
Bacterial count and body weight changes mice, for infected with Mycobacterium tuberculosis
and treated with different Streptomycin preparations

| | | | Body | Log | Log |
|---|---|---|---|---|---|
| Time | Groups | n | weight(g) | (CFU/Spleen) | (CFU/Lung) |
| D1 | Baseline (untreated group) | 5 | 16.26 ± 0.81 | 7.63 ± 0.05 | 10.06 ± 0.30 |
| D14 | Positive control | 4 | 16.22 ± 0.39 | 6.90 ± 0.08 | 7.85 ± 0.14 |
| | Comparative control | 4 | 14.90 ± 1.83 | 7.55 ± 0.26* | 8.16 ± 0.25 |
| | NP formulation (Example # 4) | 4 | 17.73 ± 1.1* | 6.03 ± 0.41* | 7.77 ± 0.08 |
| | NP formulation (Example # 3) | 4 | 18.22 ± 0.86* | 6.43 ± 0.23* | 7.10 ± 0.31* |
| D28 | Positive control | 3 | 19.62 ± 0.29 | 6.66 ± 0.28 | 7.57 ± 0.26 |
| | Comparative control | 3 | 16.73 ± 2.64 | 7.48 ± 0.51 | 8.37 ± 0.36* |
| | NP formulation (Example # 4) | 3 | 19.66 ± 3.23 | 6.26 ± 0.43 | 6.97 ± 0.16 |
| | NP formulation (Example # 3) | 4 | 20.16 ± 0.54 | 6.43 ± 0.39 | 7.18 ± 0.25 |
| D56 | Positive control | 4 | 17.85 ± 1.15 | 7.21 ± 0.23 | 8.86 ± 0.18 |
| | NP formulation (Example # 4) | 2 | 18.19 ± 5.19 | 7.04 ± 0.27 | 8.06 ± 0.51* |
| | NP formulation (Example # 3) | 4 | 20.63 ± 1.49* | 6.14 ± 0.07* | 7.33 ± 0.24* |

*P < 0.05, vs Positive control.

### Bacterial Pneumonia

An investigation of the antibacterial efficacy of nanoparticulate forms of parenterally administered Vancomycin was carried out in comparison with conventional parenteral Vancomycin sulfate for injection,

Bacteria: Staphylococcus pneumoniae (ATCC #6301), Gram-positive serotype

Animals: Male CD-1 (Charles River Laboratory) mice weighing 24±2 g provided are used. Space allocation for animals is 45×23×21 cm for 10 mice. The animals are housed in animal cages and are maintained in a hygienic environment under controlled temperature (22°-23° C.) and humidity (50% -60%) with 12 hours light/dark cycles for at least one week in the laboratory prior to initiation of the study. Free access to standard lab food and water supply is allowed.

Pneumonia model: Mice were inoculated intratracheally (IT) with a LD90-100 of Staphylococcus pneumoniae (ATCC 6301) (9.7×10⁶ CPU/mouse) in 40 μl PBS. In 16 hours, all mice developed pronounced pneumonia, confirmed by histological observation. Test formulations and vehicle were diluted with 5% sterile dextrose and administered intravenously to animals 20 hours after bacterial inoculation. Mortality was recorded once daily for 10 days.

Results are presented in table 8 and graph 4

TABLE 8
Survival rate of mice with Streptococcal lung pneumonia, treated with different Vancomycin formulations

| | Vancomycin | | Day | Day | Day | Day | Day | Day | Day | Day | Day | Day | | |
|---|---|---|---|---|---|---|---|---|---|---|---|---|---|---|
| Treatment | Dose | n | 1 | 2 | 3 | 4 | 5 | 6 | 7 | 8 | 9 | 10 | Mortality | Survival |
| Vehicle | 5 ml/kg | 10 | 0 | 0 | 0 | 4 | 4 | 2 | 0 | 0 | 0 | 0 | 100% | 0% |
| (5% Dextrose) | (no drug) | | | | | | | | | | | | | |
| Vancomycin | 1 mg/kg | 10 | 0 | 0 | 0 | 1 | 3 | 2 | 0 | 1 | 0 | 0 | 70% | 30% |
| injection (USP) | | | | | | | | | | | | | | |
| Example 10 | 1 mg/kg | 10 | 0 | 0 | 0 | 0 | 0 | 0 | 0 | 0 | 0 | 0 | 0% | 100% |
| Example 11 | 1 mg/kg | 10 | 0 | 0 | 0 | 0 | 0 | 0 | 0 | 0 | 0 | 0 | 0% | 100% |
| Example 12 | 1 mg/kg | 10 | 0 | 0 | 0 | 0 | 1 | 0 | 0 | 0 | 0 | 0 | 10% | 90% |

Results for the comparative treatment of severe lung infections with either aminoglycoside antibiotic (Streptomycin) or glycopeptide antibiotic (Vancomycin) in nanoparticle colloidal formulations, along with conventional formulations, shows that there is a significant increase in antibacterial activity in the colloidal formulations. A Streptomycin formulation according to example #3 of the invention is at least 5 times more effective than tree Streptomycin; nanoparticulate compositions of Vancomycin (examples #9 and #10) are approximately 10 times more effective than standard solutions of Vancomycin sulfate USP for injection.

Similar increases in antibacterial efficiency are observed for other antibiotics incorporated in nanoparticulate colloidal delivery systems made of biodegradable polymers (e.g., Amikacin, Kanamycin, Gentamicin, Colistin, Polymixin B, Bacitracin, fluoroquinolones).

We claim:

1. A method of treating of bacterial pneumonia and other lung infections by the systemic administration of antibiotics incorporated into biodegradable colloidal polymeric composition.

2. A pharmaceutical composition for enhanced efficacy in pneumonia treatment, comprising a plurality of biodegradable colloidal particles, loaded with water soluble antibiotic

3. A method as set forth in claim 1, wherein said colloidal polymeric composition is comprised of nanoparticles or nanocapsules.

4. A method as set forth in claim 1, wherein said antibiotic is associated with said nanoparticles or nanocapsules via non-covalent binding.

5. A biodegradable colloidal polymeric composition as set forth in claim 2, wherein said water soluble antibiotic is selected from a group of aminoglycosides and peptides.

6. A biodegradable colloidal polymeric composition as set forth in claim 3, wherein a polymer is selected from a group of biodegradable polyesters or polyester copolymers, such as biodegradable polyester polymer selected from a group comprising poly(lactic acid), poly(glycolic acid), poly(D-lactic-co-glycolic acid), poly(L-lactic-co-glycolic acid), poly(D,L-lactic-co-glycolic acid), block-copolymers of lactide, glycolide and polyethylene glycol, homo- and copolymers of polycaprolaclone, poly(hydroxybutyrate), their derivatives or mixtures thereof.

7. A biodegradable colloidal polymeric composition as set forth in claim 5, wherein nanoparticles or nanocapsules may additionally contain hydrophobic components.

8. A biodegradable colloidal polymeric composition as set forth in claim 5, which may additionally contain pharmaceutically acceptable counter-ions.

9. A biodegradable colloidal polymeric composition as set forth in claim 3, wherein the particle size of said nanoparticles or nanocapsules is in the range from 5 to 5000 nm.

10. A biodegradable colloidal polymeric composition as set forth in claim 4 wherein an antibiotic is associated with nanoparticles or nanocapsules in range from 5 to 100% of total antibiotic amount

11. A biodegradable colloidal polymeric composition as set forth in claim 7, wherein said hydrophobic components are selected from lipids, mono-, di- and triglycerides, phospholipids, waxes, sterols, tocopherol and tocopherol derivatives, aliphatic and aromatic alcohols, esters and ethers.

12. A biodegradable colloidal polymeric composition as set forth in claim 8, wherein said counter-ion molecule is selected from aliphatic acids, alkyl- or arylsulfonic acids, alkylphosphates, cholates, deoxycholates, taurocholates, phosphatidylglycerol, phosphatidylserine, aliphatic amines, lidocaine, benzocaine, benzydamine, arginine, lysine or their physiologically acceptable salts.

13. A biodegradable colloidal polymeric composition as set forth in claim 5, wherein said water soluble antibiotic is Vancomycin.

14. A biodegradable colloidal polymeric composition as set forth in claim 5, wherein said water soluble antibiotic is Colistin.

15. A biodegradable colloidal polymeric composition as set forth in claim 5, wherein said water soluble antibiotic is Polymixin.

16. A biodegradable colloidal polymeric composition as set forth in claim 5, wherein said water soluble antibiotic is Kanamycin.

17. A biodegradable colloidal polymeric composition as set. forth in claim 5, wherein said water soluble antibiotic is Streptomycin.

18. A biodegradable colloidal polymeric composition as set forth in claim 5, wherein said water soluble antibiotic is Gentamicin.

19. A biodegradable colloidal polymeric composition as set forth in claim 5, wherein said water soluble antibiotic is Amikacin.

20. A biodegradable colloidal polymeric composition as set forth in claim 12, wherein said pharmaceutically acceptable counter-ion is selected from a group of saturated and unsaturated aliphatic carboxylic acids C6-C24, aromatic acids (benzoic, terephthalic, tocopherol acid succinate, cholesteryl hemisuccinate), alkylsulfonic (lauryl sulfate, octadecyl sulfate), alkylphosphonate(cetyl- and dicetylphosphate), arylsulfonic (benzenesulfonate, naphtaline sulfonate, cholesteryl sulfonate), phospholipids (phosphatidylserine, phosphatidylglycerol).
